(19) United States
(12) Patent Application Publication
(10) Pub. No.: US 2025/0140352 A1
(43) Pub. Date: May 1, 2025

(54) SIMPLIFIED COMPOUND IDENTIFICATION

(71) Applicant: DH TECHNOLOGIES DEVELOPMENT PTE. LTD., Singapore (SG)

(72) Inventors: David Cox, Toronto (CA); Stephen Tate, Barrie (CA)

(21) Appl. No.: 18/686,627

(22) PCT Filed: Sep. 13, 2022
(86) PCT No.: PCT/IB2022/058596

§ 371 Date: Feb. 26, 2024

Related U.S. Application Data
(60) Provisional application No. 63/243,899, filed on Sep. 14, 2021.

(51) Int. Cl. G16C 20/20; H01J 49/00

(52) U.S. Cl. CPC G16C 20/20; H01J 49/0036

(57) ABSTRACT

Disclosed are systems and methods for facilitating compound identification based on analytical data obtained from an analytical instrument such as, for example, a mass spectrometer.

### RELATED APPLICATIONS

The present patent application claims the priority benefit of U.S. Provisional Patent Application Ser. No. 63/243,899, filed Sep. 14, 2021, the content of which is hereby incorporated by reference in its entirety into this disclosure.

### BACKGROUND

Methods and systems for identifying one or more unknown compounds in a sample can be difficult and resource-intensive. Accordingly, there is a need for alternative methods and systems that can identify one or more compounds that utilize one or more simplified database queries.

### SUMMARY OF THE DISCLOSURE

In an aspect, the disclosure provides a system comprising a processor configured to (i) receive mass analysis data from a compound; (ii) communicate with a network to generate one or more search queries based on the mass analysis data, wherein the one or more search queries comprises a listing of synonyms and/or common names for the compound; (iii) determine a weight averaged score based on the result of the one or more search queries; and (iv) identify the compound based on the weight averaged score.

In embodiments of the system, the processor can be configurable to generate a predicted chemical formula or molecular weight, or both, based on the mass analysis data. In some embodiments, the processor can be configurable to receive information for a compound that may include one or more of a predicted or determined chemical formula, a predicted or determined molecular weight, either or both of which may be based on analytical data such as, for example, mass analysis data.

In embodiments of the system, the processor can be configurable to generate a readability value for each synonym or common name in the listing of synonyms or common names for the compound.

In embodiments of the system, the processor can be configurable to determine the weight averaged score based on the result of the one or more search queries and the readability value for each synonym or common name.

In embodiments of the system, the processor can be configurable to generate a second listing of synonyms and/or common names for the compound that have a weight averaged score above a minimum threshold and identifies the compound from the second listing. In some further embodiments, the weight averaged core minimum threshold is above zero.

In embodiments of the system, the system may further comprise an analytical device operatively coupled to the system. In some further embodiments, the analytical device comprises a mass spectrometer.

In another aspect, the disclosure provides a method of identifying a compound comprising: receiving mass analysis data for the compound; generating one or more search queries based on the mass analysis data, wherein the one or more search queries comprises a listing of synonyms and/or common names for the compound; determining a weight averaged score based on the result of the one or more search queries; and identifying the compound based on the weight averaged score.

In another aspect, the disclosure provides a method of identifying a compound by mass analysis comprising: generating mass analysis data (e.g., a mass spectrum) for the compound; generating one or more search queries based on the mass analysis data, wherein the one or more search queries comprises a listing of synonyms and/or common names for the compound; determining a weight averaged score based on the result of the one or more search queries; and identifying the compound based on the weight averaged score.

Embodiments of the above methods may further comprise generating a predicted chemical formula or molecular weight, or both, for the compound based on the mass analysis data. In some embodiments, the above methods may further comprise receiving a predicted or determined chemical formula or molecular weight, or both, for the compound based on the mass analysis data.

Embodiments of the above methods may further comprise assigning a readability value for each synonym or common name in the listing of synonyms or common names for the compound.

In embodiments of the above methods determining the weight averaged score may be based on the result of the one or more search queries and the readability value for each synonym or common name.

Embodiments of the above methods may further comprise generating a second listing of synonyms and/or common names for the compound that have a weight averaged score above a minimum threshold. In embodiments of the above methods the weight averaged score used to identify the compound is above zero.

In embodiments of the above methods identifying the compound may comprise identifying from the second listing of synonyms and/or common names.

Other aspects and embodiments of the disclosure will be apparent in light of the description and illustrative examples that follow.

### BRIEF DESCRIPTION OF THE DRAWINGS

FIG. 1 illustrates an embodiment of a system in accordance with the disclosure that may be used in performing example embodiments of the methods as described herein.

FIG. 2 illustrates histogram comparison of methods in accordance with the aspects and embodiments of the disclosure and those using a typical rank-order method for compound identification.

FIG. 3 depicts results for a series of compounds, identified on the x-axis, and plots the rank position of the correctly identified search result as a function of the synonym-based search and the rank-order reference search.

### DETAILED DESCRIPTION

The disclosure generally provides systems and methods for identification of one or more unknown compounds based on analytical data obtained from a sample. More specifically, the methods and systems are based on one or a plurality of search queries that include synonyms or common names for the unknown compound(s) that may be generated from the analytical data. Suitably, though not necessarily in every instance, the analytical data can provide at least enough information to derive a chemical formula for the compound(s) to be identified. The chemical compound information can generate a query based on a listing of a number of names for the compound and, based on the returned results from the query, a weight averaged score can be derived that allows for the accurate identification of the compound(s).

The methods and systems can provide for simple and reliable identification of one or more unknown sample components, and improve upon existing systems and methods that are intended to aid and facilitate identification of sample components by analyzing, e.g., spectral fingerprint matching, reference/publication searching, etc., Thus, the systems and methods in accordance with the disclosure do not rely on strategies for compound identification that are based on identification and matching of spectra (e.g., fingerprint regions), which can be particularly difficult when attempting to identify novel or minimally characterized chemical entities (i.e., compounds without any spectral data in searchable databases). Instead, the systems and methods provided by the disclosure reduce the total number of query search results using a weight averaged score based on features of the results returned on the synonym or common name query which may be, for example, a readability value that is generated by a processor or other computer resources.

While various aspects and embodiments in the description that follows serve to illustrate and exemplify features of the disclosed subject matter, it is not exhaustive and does not limit the disclosure to the precise form described. Modifications and variations are possible in light of the disclosure or may be acquired from practicing one or more of the various aspects and embodiments provided herein. Additionally, aspects of the disclosure includes software but the disclosure may be implemented as a combination of hardware and software or in hardware alone. The disclosure may be implemented with both object-oriented and non-object-oriented programming systems.

The terminology used herein is for the purpose of describing particular embodiments and examples only and is not intended to be limiting of the disclosure. As used herein, the singular forms are intended to include the plural forms as well, unless the context clearly indicates otherwise. It will be further understood that the terms “comprises,” “includes,” “comprising,” “including,” “has,” “have,” “having,” and the like when used in this specification, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, these elements should not be limited by these terms. These terms are only used to distinguish one element from another element. Thus, for example, a first element, a first component or a first section discussed below could be termed a second element, a second component or a second section without departing from the teachings of the present disclosure. Similarly, various spatial terms, such as “upper,” “lower,” “side,” and the like, may be used in distinguishing one element from another element in a relative manner, and that each element may have its own structural orientation (e.g., a device may be turned sideways so that its “top” surface is facing horizontally and its “side” surface is facing vertically).

As utilized herein the terms “circuits” and “circuitry” refer to physical electronic components (i.e., hardware) and any software and/or firmware (“code”) that may configure the hardware, be executed by the hardware, and or otherwise be associated with the hardware. As used herein, for example, a particular processor and memory (e.g., a volatile or non-volatile memory device, a general computer-readable medium, etc.) may comprise a first “circuit” when executing a first one or more lines of code and may comprise a second “circuit” when executing a second one or more lines of code.

As utilized herein, circuitry is “operable” to perform a function whenever the circuitry comprises the necessary hardware and code (if any is necessary) to perform the function, regardless of whether performance of the function is disabled, or not enabled (e.g., by a user-configurable setting, factory setting, etc.).

As utilized herein, “and/or” means any one or more of the items in the list joined by “and/or”. As an example, “x and/or y” means any element of the three-element set {(x), (y), (x, y)}. That is, “x and/or y” means “one or both of x and y.” As another example, “x, y, and/or z” means any element of the seven-element set {(x), (y), (z), (x, y), (x, z), (y, z), (x, y, z)}. That is, “x, y, and/or z” means “one or more of x, y, and z.” As utilized herein, the terms “e.g.,” and “for example” set off lists of one or more non-limiting examples, instances, or illustrations.

In some aspects, the disclosure provides a method of identifying a compound comprising: receiving data for the compound such as, for example, mass analysis data; generating one or more search queries based on the data, wherein the one or more search queries comprises a listing of synonyms and/or common names for the compound; determining a weight averaged score based on the result of the one or more search queries; and identifying the compound based on the weight averaged score. In some embodiments of the method, the data for the compound to be identified is obtained from previously-acquired analytical data on the compound (i.e., an analytical measurement on a sample that is saved in one or more databases). Analytical data for one or more compounds may comprise, or may be obtained by, any known and available method. Non-limiting examples of analytical data include spectral data (e.g., light emission, light reflectance, light scattering, and light absorption (infrared (IR), near infrared (NIR), ultraviolet (UV-Vis), Raman, etc.); magnetic and electric field data (NMR, EPR, etc.); separations/mobility data (analytical chromatography, ultracentrifugation, etc.); solubility data; ionization data (mass spec)) or other analysis techniques. In some embodiments, the data for the compound is generated by an analytical instrument such as, for example, a mass spectrometer that is operatively connected to a processor or computing resources that are also operable to perform the methods of identification described herein. Thus, in some embodiments the data comprises mass data for the compound that can provide the chemical formula for the compound.

The methods and systems disclosed herein comprise generating one or more queries that comprise a listing of synonyms and/or common names for the compound and determining a weight averaged score based on the result of the one or more queries. In some embodiments the weight average score comprises determining a readability value for one or more terms that are returned by the query. As used herein, a “readability value” for a term (e.g., chemical name, chemical name, or synonym) refers to an index value that may be calculated, either manually or automated, to provide an indication of the level of education a reader is likely to require in order to be able to read the term. The calculated value can be based on one or more algorithms, formulas, and/or scoring systems and/or combination thereof. Generally, the readability value comprises an estimate of the percentage of a given population of individuals (i.e., users), at a given level of education, who can read and/or comprehend the term or terms. In some embodiments the readability value may indicate a given percent of a population (e.g., from 50-75%) having a minimum set level education level (e.g., elementary school-advanced degree). In some embodiments, the readability value comprises an estimate of the age a user would need to be in order to comprehend or pronounce the term or name. Some non-limiting factors that can be used in the calculation for determining the readability value/index value can include one or more of, the total length of the term(s)/word(s); how difficult the term/word is to pronounce; the average length of the term(s)/word(s); syllable count, number of multi-syllable terms or words, familiarity of the term(s)/word(s) (i.e., how commonly or frequently the term(s) is used); the overall complexity of the term(s)/word(s), and the like.

The systems and methods in accordance with the disclosure can comprise one or more search query. As referred to herein, the term “search query” or search queries” generally refers to a phrase or a keyword combination a user can enter in a search engine to identify and return information (e.g., answers) of relevance/interest that are contained in one or more information sources such as, for example, databases and libraries. In some embodiments a search query may be a keyword, or a combination of keywords. In some embodiments a search query may be a contextual grouping of words that may be in common usage or language.

As used herein, reference to “synonym(s)” and/or “common name(s)” generally refers to a term that may be associated with a keyword or a search query. In some embodiments a synonym or common name refers to an alternative name for the same term, e.g., a name for a chemical compound, structure, or formula, such as, for example a chemical name (e.g., IUPAC naming convention), trade or brand name, generic name, slang or “street” name, and the like. In some embodiments the synonym and/or common name is based on the chemical formula of the compound.

As used herein, a “weight averaged score” refers to an index value that may be calculated (manual or automated) to provide an indication of the relative score associated with one or more search query. In some embodiments, the weight averaged score is set to encompass results within a range of values. In some embodiments, an initial score is calculated and the initial score may be normalized by any method (e.g., a logistic function) and weighted by a value determined and/or set by a user (e.g., based on importance of the query/term).

In an example embodiment in accordance with the disclosure, a readability score of chemical names can be calculated using, e.g., the Coleman-Liau index. The score may be used to gauge or determine the understandability or complexity of text (e.g., text phrases, passages, chemical name(s)). In some embodiments the methods provide a chemical name readability value that may be calculated using a subset of chemical names and synonyms from a database (e.g., PubChem, which can comprise up to several thousand examples) and a statistical analysis of the chemical name/synonym text (e.g., the Python library, Textstat). A range of resulting values is generated (e.g., values ranging from −15 to 300), wherein simpler names such as, for example, trade names or common names are assigned a lower value range (e.g., scoring from 0-100) and more complex (e.g., IUAPAC chemical names) are assigned a higher value range (e.g., scoring above 100).

In some further example embodiments in accordance with the disclosure, the readability score can be adjusted to be a distance from the average score of most simpler trade names or common names (distance from a Coleman-Liau score of 50) and then normalized to be between 0 and 1.0. This normalized score may be combined with other scores to produce a weighted average score for compound identification. In other embodiments, the score may be converted to either 0 (for scores below 0 or above 100) or 1 (for scores between 0 and 100), which can be used as a decision criteria for performing other more resource-intensive queries and calculations (e.g,. time consuming or expensive calculations).

One non-limiting example of a resource-intensive query in accordance with embodiments of the disclosure include mass fragment pattern prediction of a chemical structure. In such embodiments a theoretical fragmentation pattern can be generated for a defined chemical structure (e.g., represented in a .mol or .sdf file) by allowing various bonds to be broken. The predicted resulting fragment m/z values are calculated using a fragmentation tool (e.g., as provided by software packages such as in PeakView 2.2). Rules for identifying bond breaks can include, for example, breaking up to 2 single bonds, 1 carbon-carbon single bond, and/or allowing ring bonds to break. A mass spectrum (e.g., MSMS) can be acquired and scored based on the percentage of fragmentation ion signal explained or predicted by the theoretical fragmentation pattern. In some embodiments a second fragmentation matching score can be determined by defining, or varying, other bond breaking rules such as, for example, breaking up to 3 single bonds, 2 carbon-carbon single bond, allowing ring bonds to break, and/or allowing bond closures/formation. These are just some non-limiting examples of the methods that are available and known in the art for calculating theoretical fragmentation patterns from a structure, and which may vary in terms of the amount of computational burden that may be required.

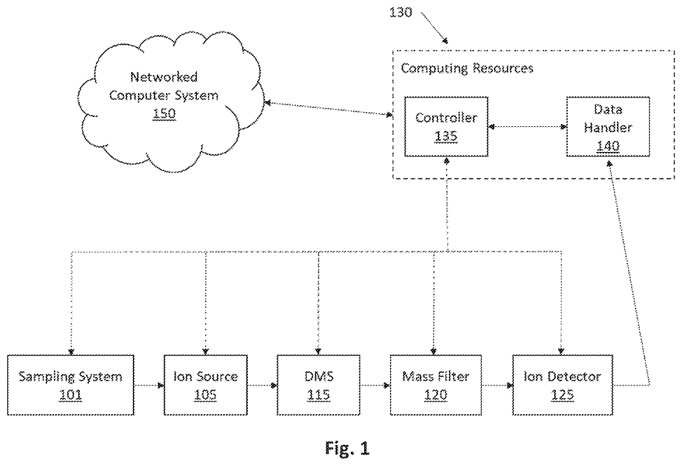

Referring to the non-limiting embodiment of the disclosure depicted in FIG. 1, a system comprising computing resources 130 can be used to implement the various methods disclosed herein. The computing resources 130 may comprise a controller 135 and data handler 140. In accordance with the aspects and embodiments of the disclosure, computing resources 130 is operably connected to one or more computer systems 150 across a network to form a networked system. The network may comprise a private network or a public network such as the Internet. In the networked system, one or more computer systems may store and serve the data to other computer systems. The one or more computer systems that store and serve the data may be referred to as servers or the cloud, in a cloud computing scenario. The one or more computer systems may include one or more web servers, for example. The other computer systems that send and receive data to and from the servers or the cloud may be referred to as client or cloud devices, for example. It will be apparent to those of skill in the relevant arts that various embodiments of the present disclosure may utilize a computer as is known in the art.

The computing resources 130 may include any suitable data computation and/or storage device or combination of such devices. An example controller may comprise one or more microprocessors working together with storage to accomplish a desired function. The controller 135 and/or data handler 140 may include at least one computing element that comprises at least one high-speed data processor adequate to execute program components for executing user and/or system-generated requests.

The computing resources 130 is configured to generate one or more search queries from the analytical data for the compound(s) to be identified, and to deliver the one or more queries to the networked computer system/databases 150. In some embodiments, the queries comprise one or more synonyms and/or common names for the compound, which can be determined from the data which is suitably a chemical formula or chemical name. For example, if the analytical data for a compound provides its chemical formula, the computing resources 130 can generate search queries based on its chemical name (e.g., IUPAC conventional name) and a series of synonyms associated with the chemical name that may include common, alternative, branded, proprietary, trade, generic, or street names. The query can be generated from, and/or submitted to, any searchable and/or accessible database or databases or global search engine on a network that contains and/or maintains collections of information regarding chemical compounds (e.g., Chemical Abstracts Service, ChemSpider, PubMed, Derwent, Google API, patent databases, structural databases, etc.).

As noted above, the returned search query results can be collected by a processor and/or computer resources 130 and are scored with a weight average in order to restrict the number of query results. The weight averaged score can be used to rank-order and identify, for example, the top results (e.g., top 50, 40, 30, 25, 20, 15, 10, 5, 4, 3, 2, or single top result) and can eliminate duplicative and “nonsense” results. Additionally or alternatively, the weight averaged score can filter results for relevance by, for example, discarding any returned results that contain irrelevant terms and information, or conversely by retaining results if they contain certain terms such as, for example, terms that have a top readability factor (e.g., can be readily understood by an expert, one of ordinary skill in the art, a layperson of average educational level, etc.).

In some further embodiments, the computer resources 130 may further comprise software that can compare and/or match analytical data with reference data. In these further embodiments, the analytical data may comprise spectra or other reference data, and can provide further identifying characteristics that can confirm the compound identity via “fingerprint comparison” or other similar methods that match/compare the analytical data. Such reference spectral libraries are available and familiar to those of skill in the art.

In some embodiments in accordance with the disclosure, the computing resources 130 may be further operable to control one or more components comprising an analytical device such as, for example, a mass spectrometer system (e.g., sampling platform, ion source, mass filters, DMS/FAIMS devices, detectors, pumps, etc.). For example, referring FIG. 1, the computer resources 130 may be operable to control circuitry that provide for one or more of: controlling sample processing (e.g., sample injection/capture, such as acoustic ejection, autosampling, etc., into, e.g., a capture fluid in a probe); controlling an ion source; applying RF and DC voltages to one or a plurality of quadrupoles for injecting ions into other regions for processing, filtering, and/or detecting; reading measurements based on the injected ions (e.g., detector outputs); and performing the methods for identifying one or more compounds as disclosed herein. In the example embodiment depicted in FIG. 1, the controller 135 may control a sampling system 101, an ion source 105, a DMS 115, a mass filter 120, and an ion detector 125. The data handler 140 may store data for processing samples, sample data, or data for analyzing sample data, and may receive an output signal from the ion detector 125.

Although some aspects and embodiments of the disclosure have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the disclosure can be implemented in hardware and/or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments in accordance with the disclosure comprise a data carrier having electronically readable control signals, which are capable of cooperating with a programmable computer system, such that one of the methods described herein is performed.

Generally, embodiments of the disclosure can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the disclosure comprises a computer program having a program code for performing one of the methods described herein, when the computer program runs on a computer.

A further embodiment of the disclosure comprises a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the disclosure comprises an apparatus as described herein comprising a processor and the storage medium.

A further embodiment of the disclosure provides a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured, or adapted, to perform one of the methods described herein.

A further embodiment comprises a computer having installed thereon the computer program for performing one of the methods described herein.

A further embodiment in accordance with the disclosure comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

While particular aspects and embodiments of the disclosure have been illustrated and described, it would be apparent to those skilled in the art that various other changes and modifications can be made and are intended to fall within the spirit and scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

### EXAMPLES

### Example 1—Identification of Cycloxaprid

Cycloxaprid is a newly developed neonicotinoid pesticide that was first listed on the Alan Wood pesticide list in July 2018 (see, e.g., Compendium of Pesticide Common Names available at alanwood.net). Access to any spectral analyses of the compound was limited. For example, prior to this study, no mass spectrum data for the compound was readily accessible to the public. Mass analysis (MS1, MSMS) was performed on the compound, returning a molecular formula of C14H15N4O3Cl by using mass accuracy and isotope peak abundance measurements and common formula prediction tools. An initial database query (ChemSpider) using a list of various synonym and names for the formula returned 594 potential compounds that were rank-ordered based on number of data sources and publications. Another query of the database (ChemSpider) made subsequently to the initial query, when sorted based on data sources and publications identifies cycloxaprid as number 286 out of 1295 (as, “(1S,8R)-5-[(6-Chloro-3-pyridinyl)methyl]-7-nitro-11-oxa-2,5-diazatricyclo [6.2.1.02.6]undec-6-ene”). When the same query is made by chemical formula and sorted by data sources, the compound first appears on page 15 of the results. To help improve the rank order of the initial results, a query assembling synonyms and from those synonyms determining a readability score for each synonyms was applied. After this reordering, only a single synonym-cycloxaprid-returned a non-zero hit score (see, Table 1).

TABLE 1
Synonym and readability rank-order results for molecular formula C14H15N4O3Cl.

| | Google | | | |
|---|---|---|---|---|
| name | Hits | Readability | msmsMatch1 | msmsMatch2 |
| cycloxaprid | 5810 | 18.4 | 0.9 | 0.9 |
| (5s,8r)-cycloxaprid | 0 | 18.4 | 0.9 | 0.9 |
| avadomide hydrochloride (usan) | 0 | 105.4 | 0.2 | 0.4 |
| uracil, 1-{p-[3-(2- | 0 | 128.6 | 0.2 | 0.2 |
| chloroethyl)ureido]benzyl}- | | | | |
| 5-chloro-n-(oxan-2-yloxy)-2-(1h-1,2,4- | 0 | 157.6 | 0.2 | 0.3 |
| triazol-1-yl)benzamide | | | | |
| 8-chloro-4-(2-methyl-1,4-oxazepan- | 0 | 175 | 0.4 | 0.4 |
| 4-yl)-6-nitroquinazoline | | | | |
| n-(1-tert-butyl-1h-pyrazol-3-yl)- | 0 | 180.8 | 0.4 | 0.6 |
| 4-chloro-2-nitrobenzamide | | | | |
| 1-(5-chloro-2-methoxyphenyl)-3-(6- | 0 | 186.6 | 0.2 | 0.3 |
| ethoxypyrimidin-4-yl)urea | | | | |
| 1-(5-chloro-2-methoxyphenyl)-3-(2-(6- | 0 | 186.6 | 0.2 | 0.3 |
| oxopyridazin-1(6h)-yl)ethyl)urea | | | | |
| 2-(7-chloro-5-nitro-1h-indazol-1-yl)- | 0 | 186.6 | 0.4 | 0.4 |
| 1- (piperidin-1-yl)ethan-1-one | | | | |
| 1-(5-chloro-2-methoxyphenyl)-3-(2-(6- | 0 | 186.6 | 0.2 | 0.3 |
| oxopyrimidin-1(6h)-yl)ethyl)urea | | | | |
| 8-chloro-4-(4-methoxypiperidin-1- | 0 | 186.6 | 0.4 | 0.4 |
| yl)-6-nitroquinazoline | | | | |
| 1-(5-chloro-2-methoxyphenyl)-3-(2- | 0 | 186.6 | 0.2 | 0.3 |
| ethoxypyrimidin-5-yl)urea | | | | |
| 4-[(6-chloropyridazin-3-yl)(2- | 0 | 186.6 | 0.4 | 0.4 |
| nitrophenyl)amino]butan-1-ol | | | | |
| 4-[(6-chloropyridazin-3-yl)(3- | 0 | 186.6 | 0.4 | 0.4 |
| nitrophenyl)amino]butan-1-ol | | | | |
| 1-(2-chloro-4-nitrophenyl)-3-[1- | 0 | 192.4 | 0.6 | 0.8 |
| (1-methyl-1h-pyrrol-3-yl)ethyl]urea | | | | |
| 1′-(4-azidobutyl)-5′-chloro-spiro[1,3- | 0 | 192.4 | 0.2 | 0.3 |
| dioxolane-2,3′-indoline]-2′-one | | | | |
| 1-(3-chloro-4-methylphenyl)-3-(2,4- | 0 | 198.2 | 0.2 | 0.4 |
| dimethoxypyrimidin-5-yl)urea | | | | |
| 4-(6-chloro-5-nitro-benzooxazol-2- | 0 | 198.2 | 0.4 | 0.6 |
| yl)-1,4-diaza-bicyclo[3.2.2]nonane | | | | |
| n-(5-chloro-2-methylphenyl)-4-(4- | 0 | 198.2 | 0.4 | 0.6 |
| nitropyrazolyl)butanamide | | | | |
| n-[3-(4-chloro-3-methyl-1h-pyrazol-1- | 0 | 198.2 | 0.4 | 0.5 |
| yl)propyl]-4-nitrobenzamide | | | | |
| 1-(2-chloro-4-methylphenyl)-3-(2,4- | 0 | 198.2 | 0.2 | 0.4 |
| dimethoxypyrimidin-5-yl)urea | | | | |
| n-(2-butylphenyl)-4-chloro-5-nitro-1h- | 0 | 198.2 | 0.4 | 0.4 |
| pyrazole-3-carboxamide | | | | |
| UPCMLD00WJUN284 | 0 | 300 | 0.2 | 0.2 |

### Example 2—Identifying Compounds Having No Entry in Spectral Library

Data is provided for a series of pesticides. Fifty three (53) of the pesticide compounds lack a reference mass spectrum. Mass analysis (MS1, MSMS) is performed on each of those 53 compounds to determine a predicted chemical formula. For each predicted molecular weight and chemical formula, a query is made to identify all compounds that have the predicted chemical formula. The results from each query result is sorted by the number of references and publications for each potential compound. In some instances, the compounds are correctly identified based on the list ranking of the number of references/publications. However, in some instances the compound is associated with very few references/publications which rank order it very low in such a list.

### Example 2A—Accurate Compound Identification by Reference Rank-Order

Mass analysis is performed on an unknown pesticide compound and assigned a molecular weight of 381.2994 and the formula C₁₈H₁₂F₅N₃₀. A query is made to return all publications and references associated with the molecular weight and/or chemical formula and returned a total of 62 results. The rank-order top return provided a total of 44 references from 32 data sources, with the next two closest results associated with 11 total references from 9 data sources and 8 references from 7 data sources, respectively. The top return identified the compound as fluxapyroxad (3-(Difluoromethyl)-1-methyl-N-(3′,4′,5′-trifluoro [1,1′-biphenyl]-2-yl)-1H-pyrazole-4-carboxamide), a broad-spectrum pyrazole-carboxamide fungicide that received approval for use in the US, Canada, and Europe as early as 2012.

### Example 2B—Failure to Identify Compound by Reference Rank-Order

Mass analysis is performed on an unknown pesticide compound and assigned a molecular weight of 359.4824 and the formula C₂₀H₂₅NO₃S. A query is made to return all publications and references associated with the molecular weight and/or chemical formula and returned a total of 2628 results. The rank-order top return provided a total of 12 references from 16 data sources. Another query of the database (ChemSpider) made subsequently to the initial query, when sorted based on data sources returned the correct compound as result 54 of 2628 results. None of the searches provided a top return that was able to correctly identify the compound; rather the compound (isofetamid) was listed among a number of results that were similarly rank-ordered, with a total of 12 data sources. Isofetamid (3-methyl-N-[2-methyl-1-(2-methyl-4-propan-2-yloxyphenyl)-1-oxopropan-2-yl]thiophene-2-carboxamide), like fluxapyroxad, is a fungicide but the majority of its publications are from 2017-2021 and later which skews the rank-order results away from an accurate identification.

### Example 3—Modified Search Improving Results

As illustrated by the prior examples, query results based only on rank-order of references/publications can fail to provide accurate identification of one or more unknown compounds in a sample. This example illustrate an improved query that increases the accuracy of the search results.

Spectral analysis (e.g., mass analysis such as MS1 and MSMS) is performed on a sample containing one or more unknown compounds to provide a molecular weight and formula. A database query is made to return a list of all matching compounds. The synonyms associated with each matching compound are retrieved. The listing of synonyms are scored based on simplicity/ease of language of the synonym (e.g., assessed based on likely education level needed to read the synonym). The synonyms having the best score based on ease of comprehension are searched in a database. These results are scored for theoretical fragmentation matching, and are sorted by hit count (most to least) to generate a rank list of potential compounds. This example illustrates the improvement in this methodology by using it to perform searches for the compounds detailed above in Examples 2A (fluxapyroxad) and 2B (isofetamid).

Using the mass analysis data to determine molecular weight and formula of the two compounds, a query is made to identify a list of synonyms for each compound. The synonyms are assembled and are assigned a readability score. Those results with the highest readability scores are searched using a general database search engine, which, in this example, utilized a Google-based internet search. As summarized in Tables 2a and 2b below, this method was able to identify both compounds as the first/highest search result return.

TABLE 2a
Fluxapyroxad

| | | | | MSMS | MSMS |
|---|---|---|---|---|---|
| | | | | match1 | match2 |
| Rank | synonym | # hits | readability | (?) | (?) |
| 1 | Fluxapyroxad | 29323 | 24.2 | 0.98 | 1 |
| 2 | JZNFJBJFKXHYMF- | 0 | 99.6 | −1 | −1 |
| | UHFFFOAYSA-N | | | | |
| 3 | — | 0 | 300 | −1 | −1 |
| 4 | — | 0 | 300 | −1 | −1 |
| 5 | — | 0 | 300 | −1 | −1 |

TABLE 2b
Isofetamid

| | | | | MSMS | MSMS |
|---|---|---|---|---|---|
| | | | | match1 | match2 |
| Rank | synonym | # hits | readability | (?) | (?) |
| 1 | isofetamid | 8350 | 12.6 | 0.98 | 1 |
| 2 | — | 0 | 300 | −1 | −1 |
| 3 | — | 0 | 300 | −1 | −1 |
| 4 | 1-(4-ethylphenylsulfonyl)- | 0 | 146 | −1 | −1 |
| | 4-phenylazepan-4-ol | | | | |
| 5 | — | 0 | 300 | −1 | −1 |

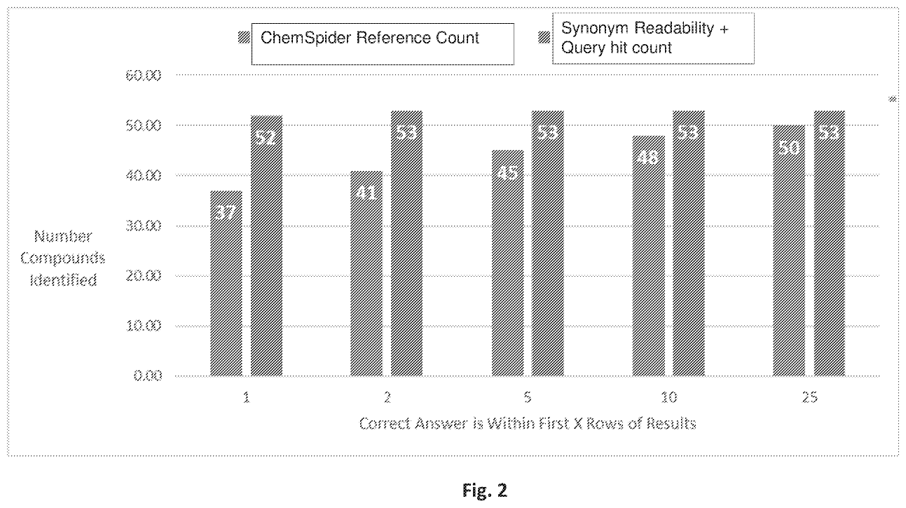

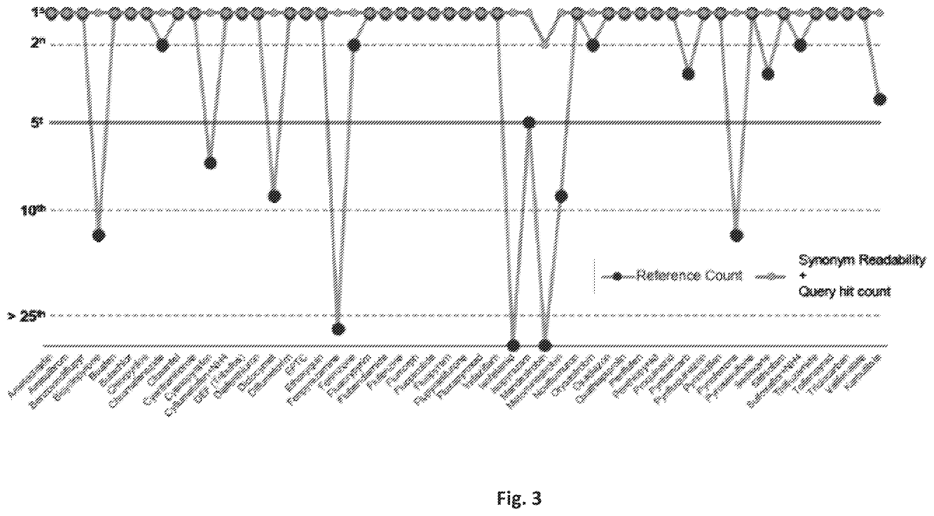

Given the success of the method, it is further validated against all 53 test compounds. The accuracy of the results are compared to the results using the rank-order publication/reference search as described in Example 2. The results are depicted in FIG. 2, which provides a histogram comparison of the results. As indicated on the x-axis, while the rank-order approach detailed in Example 2 was able to identify the correct compound as the top search result for 37 compounds. Expanding to account for the top two search results increased the number of correctly identified compounds to a total of 41. Notably, this rank-order publication method failed to correctly identify 3 of the 53 compounds even when the results were expanded to the top 25 results (FIG. 3).

Remarkably, the methodology described in Example B was able to identify 52 of 53 compounds as the top search result, and all 53 compounds were identified in the top two results.

1. A system comprising a processor configured to
(i) receive mass analysis data from a compound;
(ii) communicate with a network to generate one or more search queries based on the mass analysis data, wherein the one or more search queries comprises a listing of synonyms and/or common names for the compound;
(iii) determine a weight averaged score based on the result of the one or more search queries; and
(iv) identify the compound based on the weight averaged score.

2. The system of claim 1, wherein the processor generates a predicted chemical formula or molecular weight, or both, based on the mass analysis data.

3. The system of claim 1, wherein the processor generates a readability value for each synonym or common name in the listing of synonyms or common names for the compound.

4. The system of claim 3, wherein the processor determines the weight averaged score based on the result of the one or more search queries and the readability value for each synonym or common name.

5. The system of claim 1, wherein the processor generates a second listing of synonyms and/or common names for the compound that have a weight averaged score above a minimum threshold and identifies the compound from the second listing.

6. The system of claim 5, wherein the weight averaged core minimum threshold is above zero.

7. The system according to claim 1 further comprising a mass spectrometer.

8. A method of identifying a compound comprising
receiving mass analysis data for the compound;
generating one or more search queries based on the mass analysis data, wherein the one or more search queries comprises a listing of synonyms and/or common names for the compound;
determining a weight averaged score based on the result of the one or more search queries; and
identifying the compound based on the weight averaged score.

9. The method of claim 8, further comprising generating a predicted chemical formula or molecular weight, or both, for the compound based on the mass analysis data.

10. The method of claim 8, further comprising assigning a readability value for each synonym or common name in the listing of synonyms or common names for the compound.

11. The method of claim 10, wherein determining the weight averaged score is based on the result of the one or more search queries and the readability value for each synonym or common name.

12. The method of claim 8, further comprising generating a second listing of synonyms and/or common names for the compound that have a weight averaged score above a minimum threshold.

13. The method of claim 12, wherein identifying comprises identifying the compound from the second listing of synonyms and/or common names.

14. The method of claim 8, wherein the weight averaged score used to identify the compound is above zero.

15. A method for identifying a compound by mass spectrum analysis comprising:
generating a mass spectrum for the compound;
generating one or more search queries based on the mass spectrum, wherein the one or more search queries comprises a listing of synonyms and/or common names for the compound;
determining a weight averaged score based on the result of the one or more search queries; and
identifying the compound based on the weight averaged score.

16. The method of claim 15, further comprising generating a predicted chemical formula or molecular weight, or both, for the compound based on the mass spectrum.

17. The method of claim 15, further comprising assigning a readability value for each synonym or common name in the listing of synonyms or common names for the compound.

18. The method of claim 17, wherein determining the weight averaged score is based on the result of the one or more search queries and the readability value for each synonym or common name.

19. The method of claim 15, further comprising generating a second listing of synonyms and/or common names for the compound that have a weight averaged score above a minimum threshold.

20. The method of claim 19, wherein identifying comprises identifying the compound from the second listing of synonyms and/or common names.
